(19) United States
(12) Patent Application Publication
(10) Pub. No.: US 2025/0376738 A1
(43) Pub. Date: Dec. 11, 2025

(54) Quantitative Immuno-PCR for Simultaneous Determination of AAV Capsid Titer and AAV Genome Copy Titer

(71) Applicant: Agathos Biologics, Fargo, ND (US)

(72) Inventor: Alex Deiulio, Fargo, ND (US)

(73) Assignee: Agathos Biologics, Fargo, ND (US)

(21) Appl. No.: 19/231,189

(22) Filed: Jun. 6, 2025

Related U.S. Application Data
(60) Provisional application No. 63/657,093, filed on Jun. 6, 2024.

(51) Int. Cl. C12Q 1/70; C12Q 1/6804; C12Q 1/6851

(52) U.S. Cl. CPC C12Q 1/701; C12Q 1/6804; C12Q 1/6851; C12Q 2600/156; C12Q 2600/16

(57) ABSTRACT

Disclosed is a substrate and a method for quantitative immuno-PCR (IPCR) for simultaneous determination of AAV capsid titer and AAV genome copy titer. The substrate is an antibody to the capsid of an AAV coupled to a DNA oligonucleotide. AAV bound to the AAV antibody-DNA oligonucleotide conjugate is purified from crude or purified cell lysate using paramagnetic beads. Multiplex PCR is performed with a primer and probe set for amplification of the DNA oligonucleotide of the conjugate and a second primer and probe set for amplification of the AAV genomic sequence to determine the AAV capsid titer and AAV genome copy titer in a single reaction.

### CROSS-REFERENCE TO RELATED APPLICATIONS

This application claims priority to U.S. Provisional Patent Application No. 63/657,093, filed Jun. 6, 2024, which is incorporated by reference herein in its entirety.

### STATEMENT REGARDING GOVERNMENT FUNDING

This work was funded in part by Grant Nos. 21-283 and 23-347 from the North Dakota Department of Agriculture's Bioscience Innovation Grant Program.

### SEQUENCE LISTING

An electronic sequence listing (828349-00009.xml; size 12.0 KB; date of creation Jun. 6, 2025) submitted herewith is incorporated by reference in its entirety.

### TECHNICAL FIELD

The invention relates to the simultaneous quantitation by immuno-PCR of capsid titer and genome copy titer of recombinant AAV particles.

### BACKGROUND OF INVENTION

Genetic medicine holds great potential for correcting disease-causing defects, targeting and destroying cancerous tissues, and providing speed and flexibility for the development of vaccines. Recombinant DNA genetic material to be used as a gene therapy or a vaccine is incorporated into a virus-based vector system, which is produced by expression of the viral vector components in immortalized living cells maintained in tissue culture.

Recombinant adeno-associated virus (rAAV) vectors, which have relatively low immunogenicity, are an important platform for potential gene delivery for the treatment of a variety of human diseases. There is a need to develop clinically-useful rAAV-transgene particles, to optimize genome designs and harness the potential revolutionary biotechnologies that could contribute substantially to the growth of the gene therapy field. Preclinical and clinical successes in AAV-mediated gene replacement and gene editing have established rAAV as a promising therapeutic vector, with four AAV-based therapeutics gaining regulatory approval in Europe or the United States and more in clinical development. Continued study of AAV biology and increased understanding of the associated therapeutic challenges and limitations will build the foundation for future clinical success (see Wang, D., Tai, P. W. L. & Gao, G. Adeno-associated virus vector as a platform for gene therapy delivery. Nat. Rev. Drug. Discov. 18, 358-378 (2019)).

There is an increased demand for production of rAAV-transgene particles at high yield and at large scale for gene delivery and for protein expression and production. There is a concomitant need for improved harvesting and purification of rAAV particles, and for improved assays to determine AAV serotype identity and capsid titer, genome copy titer and genome quality (e.g., genomic integrity assays).

There are multiple methods for performing AAV capsid titer and the most widely accepted and used method is the enzyme linked immunosorbent assay (ELISA). In an ELISA to measure AAV capsid titer, AAV particles from the sample to be tested are attached to a surface such as a 96-well plate. A matching antibody to the particular AAV particle, linked to an enzyme, is applied and allowed to bind the AAV particle. Unbound antibody-enzyme conjugates are removed by a washing step(s). A substance containing the enzyme's substrate is added. If there was binding to an AAV particle, the subsequent reaction produces a detectable signal, such as fluorescence or a color change.

Quantitative polymerase chain reaction (PCR) is used to determine the genome copy titer of harvested rAAV particles. In quantitative digital PCR (dPCR), the sample is partitioned into many individual reactions so that either zero, one or more target molecules are present in each reaction, providing improved sensitivity and precision. Sample partitioning allows genomic integrity analysis of a single rAAV genome in a single partition and evaluation of amplification of specified targets. For quantitative dPCR, purified or crude lysate samples of rAAV-transgene particles are prepared using commercially-available protocols, enzymes, e.g., DNase I and Exonuclease I, and buffers as outlined in available kits, e.g., Viral Vector Lysis Kit, (Qiagen, 250272). To prime polymerization during the reaction cycles, practitioners typically use annealing primers that correlate to a portion of the promoter, polyA or transgene sequences of the particular rAAV-transgene particles being assayed. See, e.g., QIAcuity Cell and Gene Therapy (CGT) dPCR Assays (Qiagen, 250236).

In quantitative PCR, which includes qPCR, dPCR and digital droplet (ddPCR), amplification of a target DNA sequence is coupled with quantification of the concentration of that DNA species in the reaction. Quantitative qPCR, or real-time PCR (rtPCR) measures the accumulation of DNA during a PCR reaction in real time, rather than at the end of the assay. The increase in quantity of DNA at each cycle is measured by the change in intensity of a fluorescent signal. Comparison to a reference sample determines the number of original copies of template DNA in the reaction.

In quantitative dPCR or quantitative ddPCR, the sample is partitioned into many individual reactions (partitions, droplets) so that either zero, one or more target molecules are present in each reaction. The defining feature of digital PCR is the absolute quantification of nucleic acids. After partitioning, the reactions undergo end-point PCR cycling, and partitions are analyzed for the presence (positive reaction) or absence (negative reaction) of a fluorescence signal. Based on this information, one can calculate the absolute number of molecules present in the sample. Unlike qPCR, dPCR does not rely on standard curves. Consequently, dPCR has a lower detection limit and higher precision than qPCR. See www.qiagen.com/us/applications/digital-pcr?cmpid=CM_PCR_dPCR_Traffic_0123_SEA_GA_NA&gad_source=1&gclid=EAIaI QobChMIw8SoraHvhAMVmOtHAR2W-AdjEAAYAiAAEgJ7ifD_BwE.

Quantitative PCR is used to determine the genome copy titer of rAAV-transgene particles. The particles may be harvested and purified by known methods. For quantitative PCR of rAAV-transgene particles, the practitioner may use known protocols, enzymes and buffers included in commercially-available kits, e.g., QIAcuity Probe PCR Kit (Qiagen, 250102). Quantitative PCR is used to amplify a target sequence and quantitate the genome copy titer of harvested rAAV-transgene particles. Small amounts of the nucleic acid of the rAAV-transgene particles are recognized and bound during denaturation-renaturation cycles by an annealing primer that is chosen by the practitioner to have a sequence that is anti-sense to that of a promoter, poly(A) or transgene found in the particular rAAV-transgene particle being assayed. The annealing primer is recognized by the polymerase that duplicates the nucleic acid during the polymerization step of the cycle and thereby amplifies the nucleic acid of the rAAV-transgene particle.

The necessity of determining AAV capsid titer and AAV genome copy titer in two separate assays, an ELISA-based assay for capsid titer and quantitative PCR for genome copy titer, is costly and time consuming. Further, the ELISA-based assay suffers from lack of sensitivity. There is a need to develop a process whereby AAV capsid titer and genome copy titer can be determined in one single assay with improved sensitivity.

### DETAILED DESCRIPTION OF THE DRAWINGS

The present disclosure can be better understood, by way of example only, with reference to the following drawings. The elements of the drawings are not necessarily to scale relative to each other, emphasis instead being placed upon clearly illustrating the principles of the disclosure.

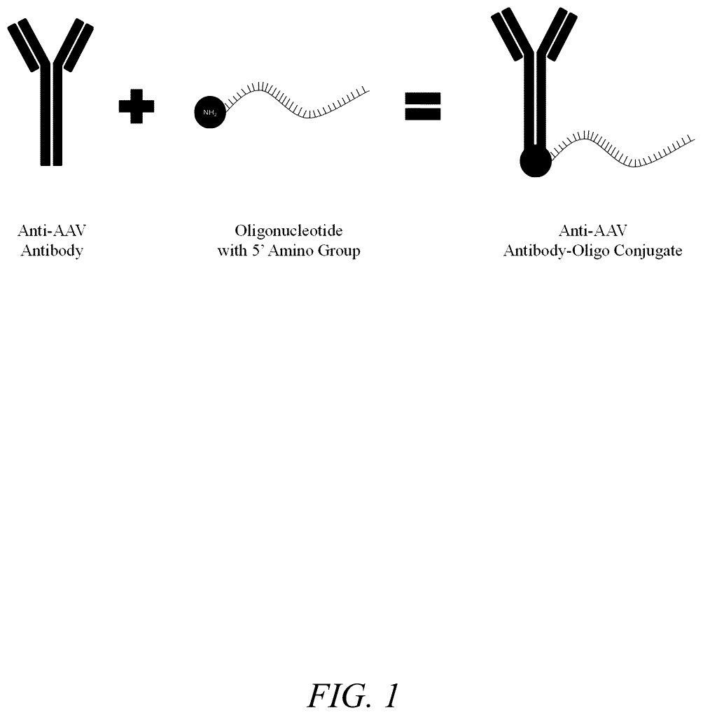

FIG. 1 is a schematic representation of a conjugation reaction in which an antibody to a capsid of an adeno-associated virus (AAV), labelled “Anti-AAV Antibody”, is coupled to a single-stranded DNA oligonucleotide having a 5′ amino group, labelled “Oligonucleotide with 5′ Amino Group”, to form an “Anti-AAV Antibody-Oligo Conjugate”.

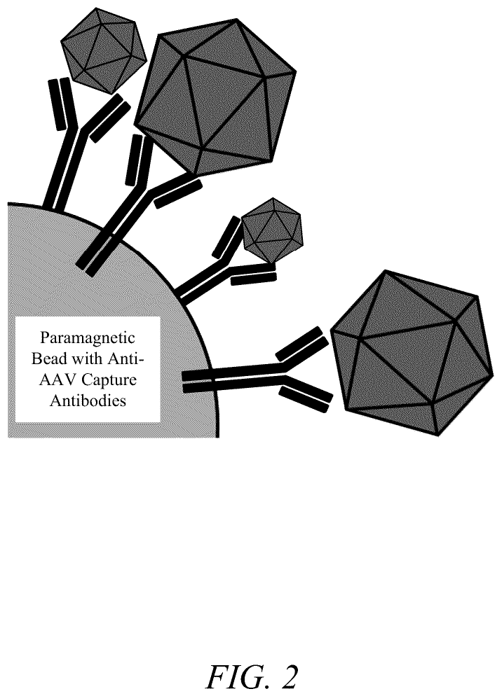

FIG. 2 is a schematic representation of an “AAV Pulldown” in which Anti-AAV Antibodies are attached to a paramagnetic bead to capture AAVs of interest.

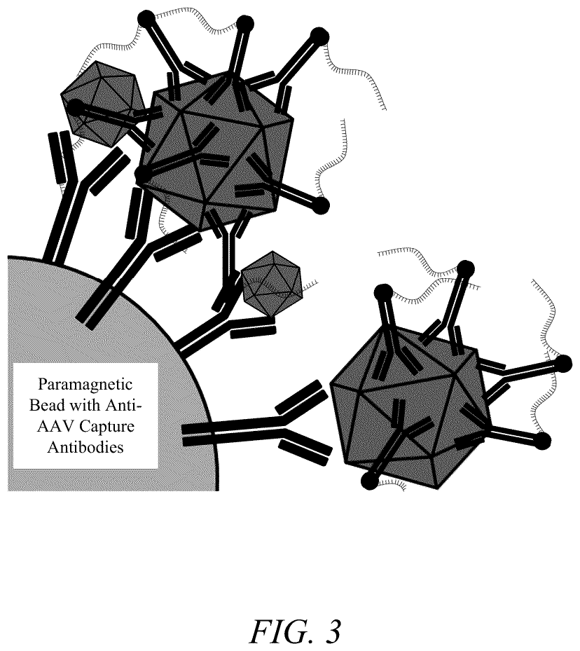

FIG. 3 is a schematic representation of “Immunostaining” in which the captured AAVs of interest of FIG. 2 are treated with the “Anti-AAV Antibody-Oligo Conjugates” of FIG. 1 and those Conjugates attach to the captured AAVs of interest.

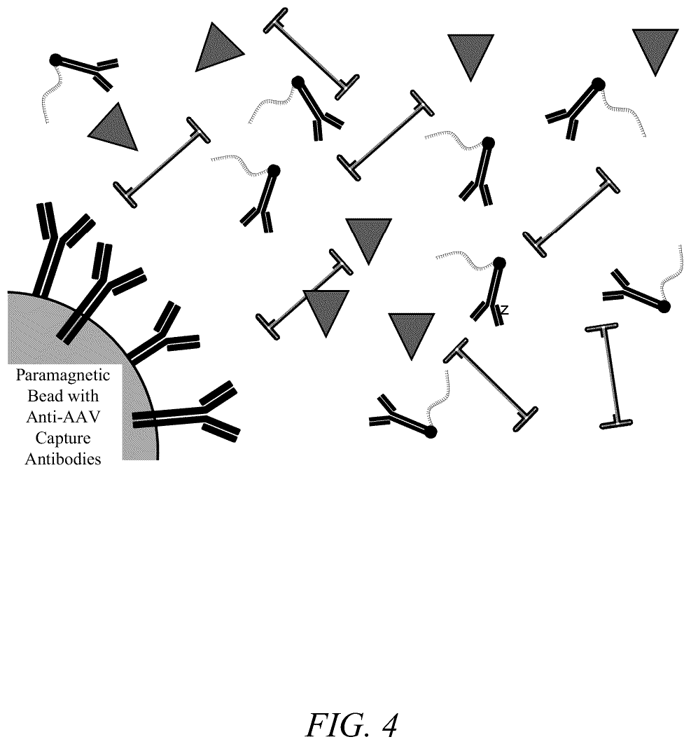

FIG. 4 is a schematic representation of “Thermal Capsid Denaturation” in which the captured AAVs of interest are denatured by treatment with heat, resulting in release of the previously bound Anti-AAV Antibody-Oligo Conjugates and the genomic sequence of the captured AAVs. The triangles represent capsids of AAVs and the linear bars represent the genomic sequences of AAVs.

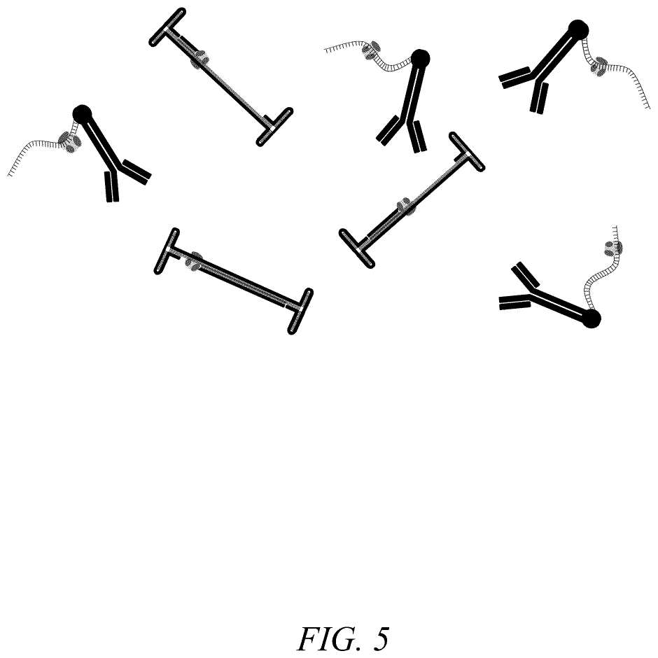

FIG. 5 is a schematic representation of quantitative multiplex PCR performed with a primer and probe pair for amplification of the oligonucleotide of the Antibody-Oligo conjugate and a second primer and probe pair for amplification of the AAV genomic sequence to quantitate the AAV capsid titer and AAV genome copy titer in a single reaction. The linear bars represent the genomic sequences of AAVs. The short bars bound to the AAV genomic sequences represent the DNA polymerase of the polymerase chain reaction.

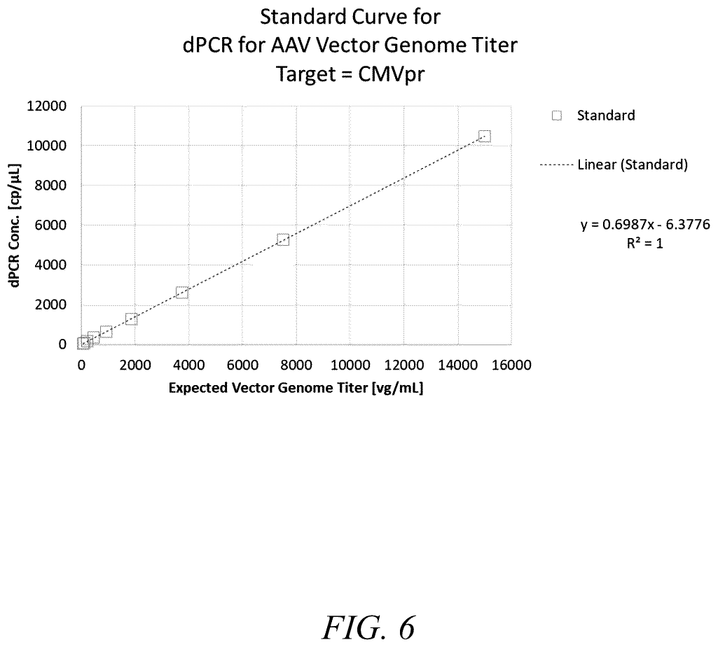

FIG. 6 is a graphical representation of a standard curve for dPCR for AAV vector genome titer for target CMVpr and was prepared from quantifying the CMVpr of known standards.

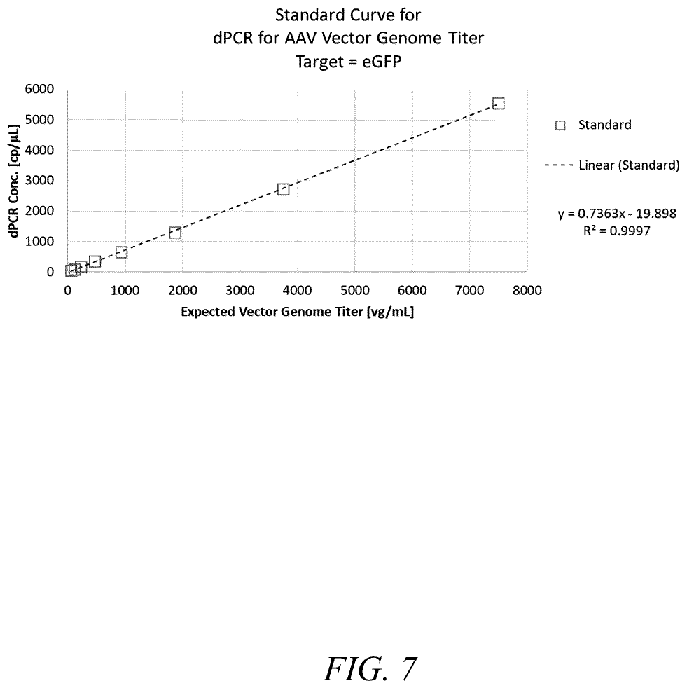

FIG. 7 is a graphical representation of a standard curve for dPCR for AAV vector genome titer for target eGFP and was prepared from quantifying the eGFP of known standards.

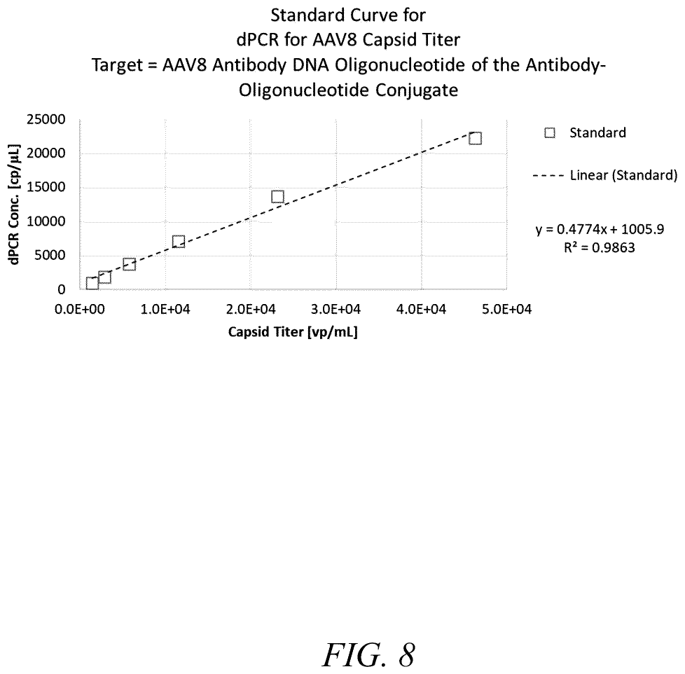

FIG. 8 is a graphical representation of a standard curve for dPCR for AAV8 capsid titer for target AAV8 Antibody DNA Oligonucleotide of the Antibody-Oligonucleotide Conjugate prepared by plotting known capsid titer values to Ct values.

### DETAILED DESCRIPTION OF THE INVENTION

Embodiments described herein can be understood more readily by reference to the following detailed description. Elements, apparatus and methods described herein, however, are not limited to the specific embodiments presented in the detailed description and examples. It should be recognized that these embodiments are merely illustrative of the principles of the present disclosure. Numerous modifications and adaptations will be readily apparent to those of skill in the art without departing from the spirit and scope of the disclosure.

There are multiple methods for quantitating AAV capsid titer and the most widely accepted and used method is the enzyme linked immunosorbent assay (ELISA). Immuno-PCR (IPCR) was developed for protein quantification to overcome the limitations of the ELISA-based assay, which is expensive, time consuming, and lacks sensitivity. The IPCR assay combines the versatility and robustness of immunoassays with the exponential signal amplification power of the polymerase chain reaction (PCR). Typically, IPCR allows a 10- to 1000-fold increase in sensitivity over the analogous ELISA. This is achieved by replacing the signal-producing antibody-enzyme conjugate of an ELISA with an antibody-DNA oligonucleotide conjugate in which the oligonucleotide serves as a target for PCR amplification. The amplification power of the PCR allows for the detection of even single molecules of nucleic acid templates. IPCR, however, has not been used to measure capsid titer of AAV. Moreover, there is no conventional method for simultaneously determining AAV capsid titer and AAV genome copy titer in a single reaction.

The invention includes new compositions of matter and new methods for the simultaneous determination of capsid titer and genome copy titer of AAV particles including recombinant AAV (rAAV) particles. The invention uses the principles of IPCR, in which the signal-producing antibody-enzyme conjugate of an ELISA is replaced with an antibody-DNA oligonucleotide conjugate. The antibody of the antibody-oligonucleotide conjugate has specificity to a capsid of an AAV of interest. The DNA oligonucleotide serves as a target for a primer and probe set for PCR amplification. The genomic sequence of the AAV, carrying a transgene of interest in rAAV, serves as a target for a second primer and probe set for PCR amplification. Multiplex quantitative PCR, which can be qPCR (also called real-time PCR (rtPCR)), digital PCR (dPCR) or droplet digital PCR (ddPCR), is used to quantitate AAV capsid titer and AAV genome copy titer in the same reaction.

A novel feature of this invention is an anti-AAVX antibody (the “X” designates that the AAV may be of any serotype) coupled to a DNA oligonucleotide. The preparation of the AAV antibody-oligonucleotide conjugate is schematically represented in FIG. 1. In one embodiment, the anti-AAVX antibody-DNA oligonucleotide conjugate is prepared using the Oligonucleotide Conjugation Kit (ab218260) from Abcam: Oligonucleotide Conjugation Kit/Easy Oligonucleotide Labeling (ab218260) (abcam.com). However, other commercial conjugation methods and kits are possible.

The DNA oligonucleotide is designed to couple with an anti-AAVX antibody. An amino group is added to the 5′ end of the oligonucleotide prior to conjugation. In the conjugation reaction, the 5′ amine group is conjugated to free amines on the antibody (usually at the lysine residues of the N-terminal). The DNA oligonucleotide may include an appropriate restriction enzyme cleavage site for separation of the DNA oligonucleotide from the antibody. In one embodiment, the restriction site is at the 5′ end of the oligonucleotide, which is the same end as the amino group. In a preferred embodiment, the restriction site starts one nucleotide away from the amino group. However, spacing of the restriction site from the amino group can be increased or decreased as desired. A preferred restriction site is MspI because MspI restriction endonuclease also is used to digest MspI sites in the inverted terminal repeats (ITRs) of the AAV vector genome to allow for better access to the AAV genomic sequence for PCR. However, the restriction site may be altered based on the desired application and other restriction sites are possible. The DNA oligonucleotide may be double-stranded or single-stranded. For constructs using a single-stranded DNA oligonucleotide, a restriction site for a restriction endonuclease that cleaves single-stranded DNA may be included. In a preferred embodiment, the DNA oligonucleotide is double-stranded and the sequence of the 5′ to 3′ strand is SEQ ID NO: 1, /5AmMC6/ACCGGCATGTGCACCGTGAAGTCCTCCGCTCCCCCCTACAAAGAC GATAAGCTAGCAGCATTGCAAGGATCGGCGATATTAACAGAGAT. “5AmMC6” denotes the 5′ amino group that attached to the AAV antibody. The MspI restriction site is “CCGG” at nucleotides 2-5 of the oligonucleotide. The double-stranded DNA is represented by the single sequence of SEQ ID NO: 1, which is fully complementary to its second strand.

An AAV-containing sample (crude cell lysate or purified lysate) of any serotype is mixed with anti-AAVX paramagnetic beads and the AAV is “pulled down”, meaning affinity purified as schematically represented in FIG. 2. The anti-AAVX paramagnetic beads comprise paramagnetic beads to which antibodies to the capsids of AAV are attached. In one embodiment, the paramagnetic beads are DynaBeads™ (Thermo Fischer Scientific), which are superparamagnetic spherical polymer particles with a uniform size and a consistent, defined surface for the adsorption or coupling of various bioreactive molecules or cells.

In one embodiment, the anti-AAV antibody is an AAV8 capsid mouse monoclonal antibody from Progen. This antibody is referred to as “anti-AAV8 (intact particle) mouse monoclonal, ADK8 antibody”. Although referred to as an anti-AAV8 antibody, this antibody specifically reacts with AAV1, AAV3, AAV7, AAV8, AAVrh10, AAVrh74 and Anc80 (both empty and full capsids). The predicted binding site is residues 586-LQQQNTO-591. Gurda et al. (2012) pubmed.ncbi.nlm.nih.gov/22593150/) showed that the ADK8 binding site is formed by an AAV variable region, VRVIII (amino acids 586 to 591 [AAV8 VP1 numbering]) that lies on the surface of the protrusions facing the 3-fold axis. This can be purchased from Progen: us.progen.com//anti-AAV8-intact-particle-mouse-monoclonal-ADK8-lyophilized-purified/610160.

The anti-AAVX antibody-DNA oligonucleotide conjugate schematically represented in FIG. 1 is mixed with the AAVX-paramagnetic bead slurry schematically represented in FIG. 2 and the anti-AAVX antibody-DNA oligonucleotide conjugate binds to the AAV as schematically represented in FIG. 3. This mixture is pulled down and rinsed multiple times to wash away unbound antibody.

Heat is applied to the mixture to denature the AAV capsids, which results in the release of the previously bound Anti-AAV antibody-oligonucleotide conjugates and the genomic sequences of the captured AAVs as schematically represented in FIG. 4. In one embodiment, the DNA oligonucleotide is cleaved from the AAV antibody by a restriction endonuclease.

Both the DNA oligonucleotide and the AAV genome are templates in a multiplexed qPCR, dPCR or ddPCR reaction as schematically represented in FIG. 5. Separate probe and primer sets are used to amplify the DNA oligonucleotide and the AAV genome. Multiple primer and probe sets for the AAV vector genome could be used to determine if the vector genome of the rAAV of interest is full or truncated. The novel process of this invention uses a quantitative PCR instrument to quantify simultaneously AAV capsid titer and AAV genome copy titer in the same reaction. In one embodiment, AAV capsid titer and genome copy titer are quantitated absolutely.

In another embodiment, a serial dilution of AAV with known capsid titer is used to create a standard curve for capsid titer. A standard curve may be helpful for at least three reasons. Each conjugation reaction could have a different amount of oligonucleotide conjugated to each antibody and a standard curve would account for that variability without having to establish a coefficient for the lot. Second, because each AAV has as many as twenty binding sites for a specific AAV antibody, there could be more than one antibody binding to each AAV particle and a standard curve will control for that variability. Third, there is innately some variability during antibody binding, both during AAV capture and during immunostaining with the antibody-oligonucleotide conjugate, and a standard curve will control for that variability.

In other embodiments, the conditions of the “pull down” affinity purification using paramagnetic beads may be adjusted to concentrate the AAV instead of merely purifying the sample. The use of a paramagnetic bead AAV “pulldown” provides a purification step so that inhibition or interference to the assay from the background matrix is minimized, which results in improved precision. The purification step also can concentrate low titer samples and thereby increase sensitivity. Increased sensitivity provides the additional benefit of using less sample to measure both AAV capsid and genome copy titers.

In one embodiment, the AAV capsid titer assay using the DNA oligonucleotide as a target for PCR may be performed independently of the AAV vector genome copy assay. Additional PCR assays can be performed in multiplex using additional primer and probe sets to identify other DNA of interest including residual host cell DNA, plasmid DNA and mycoplasma.

A “vector” is a nucleic acid molecule, a plasmid, virus (e.g., AAV vector), or other vehicle that can be manipulated by insertion or incorporation of a nucleic acid. A viral vector is derived from or based upon one or more nucleic acid elements that comprise a viral genome. The term “recombinant,” as a modifier of vector, such as recombinant AAV vector, as well as a modifier of sequences such as recombinant polynucleotides and polypeptides, means that the compositions have been manipulated (i.e., engineered by recombining genetic sequences) using molecular biology techniques into a form that generally does not occur in nature.

Adeno-associated virus (AAV) is a small (approximately 25 nm), non-enveloped virus of the Parvoviridae family, including twelve (12) different AAV serotypes, that infects humans and some other primate species. They are replication-deficient and in nature have linear single-stranded DNA (ssDNA) genomes. The wild-type AAV genome is about 4.7 kb and contains rep and cap coding sequences, flanked by ITRs. A “recombinant AAV (rAAV) vector” is derived from the wild type (wt) genome of AAV by using molecular methods to remove all or a portion of the wild-type genome from the AAV genome, for example, the rep/cap genes, and replacing it with a non-native nucleic acid sequence, referred to as a heterologous nucleic acid or transgene. Typically, one or both iTR sequences of the AAV genome are retained and flank the cloned non-native sequence in the AAV vector.

### Sequences

Table 1 identifies examples of the nucleotide sequences of the present disclosure.

TABLE 1
Identities of nucleic acid sequences of the present disclosure.

| Sequence Name | Sequence Identifier |
|---|---|
| DNA Oligonucleotide of the Antibody-Oligonucleotide Conjugate | SEQ ID NO: 1 |
| Forward Primer for PCR of the Oligonucleotide Conjugate | SEQ ID NO: 2 |
| Reverse Primer for PCR of the Oligonucleotide Conjugate | SEQ ID NO: 3 |
| Probe for PCR of the Oligonucleotide Conjugate | SEQ ID NO: 4 |
| Forward Primer for PCR of the AAV Genome CMV region | SEQ ID NO: 5 |
| Reverse Primer for PCR of the AAV Genome CMV region | SEQ ID NO: 6 |
| Probe for PCR of the AAV Genome CMV region | SEQ ID NO: 7 |
| Forward Primer for PCR of the AAV Genome eGFP region | SEQ ID NO: 8 |
| Reverse Primer for PCR of the AAV Genome eGFP region | SEQ ID NO: 9 |
| Probe for PCR of the AAV Genome eGFP region | SEQ ID NO: 10 |

Table 2 displays the nucleotide sequences identified as SEQ TD NO: 1 through SEQ ID NO: 10.

TABLE 2
Sequences of nucleic acids of
the present disclosure.

| Sequence | |
|---|---|
| Identifier | Sequence (5′ to 3′) |
| SEQ ID NO: 1 | /5AmMC6/ACCGGCATGTGCACCGTGAAGTCC |
| | TCCGCTCCCCCCTACAAAGACGATAAGCTAG |
| | CAGCATTGCAAGGATCGGCGATATTAACAGAGAT |
| SEQ ID NO: 2 | GTGCACCGTGAAGTCCTC |
| SEQ ID NO: 3 | CTCTGTTAATATCGCCGATCC |
| SEQ ID NO: 4 | CCCCTACAAAGACGATAAGCTAGCAGCA |
| SEQ ID NO: 5 | TTCCTACTTGGCAGTACATCTACG |
| SEQ ID NO: 6 | GTCAATGGGGTGGAGACTTGG |
| SEQ ID NO: 7 | TGGGCGTGGATAGCGGTTTGACTCA |
| SEQ ID NO: 8 | CAACAGCCACAACGTCTATATCAT |
| SEQ ID NO: 9 | ATGTTGTGGCGGATCTTGAAG |
| SEQ ID NO: 10 | CCGACAAGCAGAAGAACGGCATCAA |

### Example 1

### Conjugation of a DNA Oligonucleotide to an Anti-AAV Antibody (FIG. 1)

The DNA oligonucleotide (SEQ TD NO: 1) used for conjugation to an anti-AAV antibody was synthesized de novo by Integrated DNA Technologies with the 5′ amino modifier C6, HPLC-purified and lyophilized. A mouse monoclonal antibody to AAV8 capsids was obtained from Progen: us.progen.com//anti-AAV8-intact-particle-mouse-monoclonal-ADK8-lyophilized-purified/610160.

For handling, storage and resuspension of the DNA oligonucleotide and the antibody, the specific guidelines provided by the manufacturers were followed. Briefly, 1 mL of 1×PBS was added to the antibody to reconstitute. This was pipetted gently to resuspend without vortexing. The DNA oligonucleotide was resuspended in 1×PBS to reconstitute. The mixture was incubated for 20 minutes at room temperature before gentle vortexing to resuspend the DNA oligonucleotide.

For conjugation we used the Abcam Oligonucleotide Conjugation Kit (#AB218260). The control antibody and control DNA oligonucleotide provided in the kit were used as recommended. The control antibody and control DNA oligonucleotide were resuspended according to the instructions provided by the manufacturers. Briefly, we added 100 μL of Wash Buffer provided in the kit to one vial of control oligonucleotide and one vial of control antibody. Both vials were mixed gently and incubated for 30 minutes at room temperature.

The DNA oligonucleotides (SEQ ID NO: 1 and the control oligonucleotide) were activated using the protocol provided by the conjugation kit. Briefly, we added 100 μL of each oligonucleotide to the Oligo Activation Reagent vials provided in the kit. Each vial was mixed gently and incubated for 30 minutes at room temperature.

The antibodies (sample and control) were activated using the protocol provided by the kit. Briefly, we added 100 μL of each antibody (at 1 mg/mL concentration) to the Antibody Activation Reagent vials. Each vial was then pipetted gently to mix and incubated for 30 minutes at room temperature.

To desalt the activated antibodies and DNA oligonucleotides, we used one single-use Separating Column provided in the kit for each activated DNA oligonucleotide and activated antibody to be desalted. Briefly, the Separating Columns were secured in a vertical position over a collection tube. The upper and lower caps were removed to allow storage liquid to flow through. This flow through was discarded. Each column was equilibrated by adding 3 mL of Wash Buffer to the top of the column and allowing it to drip through with gravity. The flow through was discarded and the wash was repeated four times for a total of five washes.

We then added 100 μL of activated oligonucleotide or antibody to the top of the column and allowed the liquid to absorb completely into the column. Any flow through was collected and kept until successful conjugation was confirmed. We added 550 μL of Wash Buffer to the top of the column. This pushed the activated material to the bottom of the column. We waited until this liquid absorbed completely before proceeding to the next step. The flow through was collected and kept until confirmation of a successful conjugation. A clean microcentrifuge tube was placed under each column. We added 300 μL of Wash Buffer to the top of the column to elute. The eluate was collected from the column until the entire eluate was collected. This column eluate (300 μL) contained activated oligonucleotide or activated antibody that was ready for use in the conjugation.

An aliquot of the activated oligonucleotide was stored at 20° C. for long term storage. The remaining activated oligonucleotide was stored on ice until use and then at 20° C. The activated antibody was stored on ice and was used within 2 hours as per the kit protocol. It is generally not stable enough to be stored for longer.

A range of antibody:oligonucleotide ratios were made by serially decreasing the amount of oligonucleotide in the mixtures. The conjugation mixtures were incubated at room temperature for 1 hour. Alternatively, the kit provides that the conjugation reactions may be incubated overnight at room temperature with no expected adverse effect.

To purify the antibody-DNA oligonucleotide conjugate the Conjugate Clean Up Reagent provided by the kit was dissolved by placing the tube in a warm bath (37° C.) for 10 minutes and with regular mixing. An equal volume of Conjugate Clean Up Reagent was added to each antibody-oligo conjugate. These were mixed by pipette and incubated on ice for 20 minutes. After incubation we centrifuged each tube in a benchtop microcentrifuge for five minutes at 15,000×g. The tubes were removed from the microcentrifuge carefully. The supernatant was carefully removed and kept until efficient precipitation was confirmed. We added 100 μL of the Antibody Suspension Buffer to the pellet and mixed with a pipette gently to resuspend. To remove as much free oligonucleotide as possible, this was repeated for a total of two purifications. The antibody-DNA oligonucleotide conjugate was now ready for use. Each antibody-DNA oligonucleotide conjugate was mixed 1:1 with 100% glycerol and stored at −20° C.

The presence of the antibody-DNA oligonucleotide conjugate was confirmed by electrophoresis on a reducing SDS-PAGE gel. A small amount (5 μL) of the conjugate (and unconjugated antibody and oligonucleotide controls diluted to the same concentration) were mixed 1:1 with 2× gel loading buffer and heated at 95° C. for 15 minutes. After cooling the samples to room temperature, 10 μL was loaded into a 4-12% Bis-Tris SDS-PAGE gel and run for an hour at 120V. After the run, the gel was added to a container of InstantBlue Coomassie and stained for one hour. After the staining was done, the gel was added to deionized water for overnight destaining. In addition to qualitative SDS-PAGE, the recovery of antibody-oligonucleotide conjugate was quantitated using the BCA assay. In addition to protein quantitation by BCA assay, the purified antibody-oligonucleotide conjugate was used in a dPCR assay to determine the ratio of antibody to oligonucleotide that was achieved.

### Example 2

### AAV Pulldown with AAVX Paramagnetic Beads (FIG. 2)

We followed the instructions of the manufacturer of paramagnetic beads to perform the AAV pulldown. Briefly, we placed 40 μL of slurry of DynaBeads (Thermo Fischer Scientific) into a 1.5 mL microcentrifuge tube. We added 460 μL of Binding/Wash buffer (1×PBS) to the beads and gently vortex. The tubes were placed into a magnetic stand to collect the beads. The supernatant was removed and discarded. We added 500 μL of Binding/Wash buffer to each tube and incubated at room temperature for 5 minutes. The tubes were added to a magnetic stand to collect the beads. The supernatant was removed and discarded. We added 500 μL of each sample and standard to the washed beads and gently pipetted to mix. The samples were incubated at room temperature with mixing at 300 RPM on a rotating platform for 30 minutes. Tubes were then moved into a magnetic stand to collect the beads. The supernatant was removed and discarded. We added 500 μL of Binding/Wash Buffer to the tube and mixed by pipetting. The tubes were added to a magnetic stand to collect beads. The supernatant was removed and discarded. This wash step was repeated twice more for a total of three washes.

### Example 3

### Immunostaining with the Antibody-Oligonucleotide Conjugate (FIG. 3)

A 1:200 dilution of the anti-AAV8 antibody-oligonucleotide conjugate was made. We added 500 μL of the diluted anti-AAV8 antibody-oligo conjugate to each tube and was pipetted to mix. This mixture was incubated for 30 minutes at room temperature with gentle mixing. Tubes were then placed into a magnetic stand to collect the beads. The supernatant was removed and discarded. We added 500 μL of Binding/Wash Buffer to the tube and pipetted to mix. Tubes were added to a magnetic stand to collect beads. Supernatant was removed and discarded. The above wash step was repeated twice for a total of three washes.

### Example 4

### Thermal Capsid Denaturation to Release AAV Genome and the Antibody-Oligonucleotide Conjugate (FIG. 4)

The sample tubes were added to a magnetic stand to collect the beads. The supernatant was removed and discarded. We added 100 μL of qPCR Proteinase K Buffer (Teknova 2P0355) and the beads were pipetted to mix. The plate was covered with PCR film and added to a thermal cycler for a 15-minute incubation at 95° C., followed by cooling to 4° C. After the run, the denatured samples were stored on ice.

### Example 5

### Multiplex Quantitative PCR (FIG. 5)

Multiplex quantitative PCR was performed on the denatured samples using dPCR. Briefly, a PCR dilution buffer was prepared. A dPCR duplex master mix was prepared using a primer and probe set that targets the DNA oligonucleotide sequence (SEQ ID NO: 2 for forward primer; SEQ ID NO: 3 for reverse primer; SEQ ID NO: 4 for probe), a primer and probe set that targets the CMV promoter (CMVpr) region of the AAV vector genome (SEQ ID NO: 5 for forward primer; SEQ ID NO: 6 for reverse primer; SEQ ID NO: 7 for probe), and a primer and probe set that targets the eGFP region of the AAV vector genome (SEQ ID NO: 8 for forward primer; SEQ ID NO: 9 for reverse primer; SEQ ID NO: 10 for probe). The probe that targets the DNA oligonucleotide sequence is a probe modified with a HEX fluorophore used with Iowa black quencher. The probe that targets the CMV promoter of the AAV vector genome is a probe modified with a FAM fluorophore used with Iowa black quencher. The probe that targets the eGFP region of the AAV vector genome is a probe modified with a TAMRA fluorophore used with Iowa black quencher. Any appropriate compatible fluorophore could be used in place of HEX, FAM or TAMRA. Samples were transferred to a 96-well PCR plate. The samples were diluted 100-fold into dPCR dilution buffer. A four-point ten-fold dilution series starting at 200× was made. Sample dilutions were added to the dPCR master mix in duplicate. The reactions were pipetted to mix and transferred into the final reaction plate. The manufacturer's default partitioning parameters were used. The thermal cycler profile included a 15-minute 95° C. initial denaturation, followed by 40 cycles of 15 seconds at 95° C. followed by 30 seconds at 60° C. A person of ordinary skill in the art would also be able to perform multiplex quantitative PCR using qPCR or ddPCR and following known protocols.

### Example 6

### Data Analysis for AAV Genome Copy Titer and AAV Capsid Titer

Multiplex quantitative PCR was performed on the denatured samples using dPCR as described in Example 5. Genome copy titer was determined by absolute quantification and relative quantification. For relative quantification, an AAV standard containing both genome sequence targets (CMVpr and eGFP) was used to create a standard curve by plotting known vector genome titer values to the cycle threshold (“Ct”) values. Cycle threshold is the auto-threshold that the QIAcuity dPCR machine sets based on separation of positive and negative partitions. The number of positive partitions is then used to calculate the cp/μL of the dPCR reaction. The genome copy titer of samples of interest was determined by comparison to this standard curve. For absolute quantification, the raw copy/μL result from the digital PCR was used as the genome copy titer of samples of interest without any calibration by a standard curve.

Table 3 shows the absolute quantification of AAV Vector Genome Titer determined by detecting and quantifying CMVpr. Samples of unknown amount were quantified along with samples with known amounts of CMVpr as a positive control.

TABLE 3
AAV vector genome titer absolute quantification using CMVpr.

| | | AAV Vector Genome Titer for CMVpr | | | | | | | |
|---|---|---|---|---|---|---|---|---|---|
| | | | Expected | | | Average | | | |
| | | Conc. | Vector | | Vector | Vector | | | |
| Sample | | [cp/μL] | Genome | | Genome | Genome | Standard | | AVG. |
| Information | | (dPCR | Titer | Accuracy | Titer | Titer | Deviation | Precision | Accuracy |
| Function | Well | reaction) | [vg/mL] | (% REC) | [vg/mL] | [vg/mL] | [vg/mL] | (% CV) | (% REC) |
| Known | C3 | 10468 | 15000 | 70% | 8.4E+12 | 8.3E+12 | 3.6E+11 | 4% | 69% |
| | C4 | 5254 | 7500 | 70% | 8.4E+12 | | | | |
| | C5 | 2615 | 3750 | 70% | 8.4E+12 | | | | |
| | C6 | 1262 | 1875 | 67% | 8.1E+12 | | | | |
| | C7 | 629 | 938 | 67% | 8.1E+12 | | | | |
| | C8 | 348 | 469 | 74% | 8.9E+12 | | | | |
| | C9 | 169 | 234 | 72% | 8.7E+12 | | | | |
| | C10 | 75 | 117 | 64% | 7.7E+12 | | | | |
| | C11 | 41 | 59 | 70% | 8.3E+12 | | | | |
| Unknown | G3 | 10790 | N/A | N/A | 8.6E+12 | 8.7E+12 | 5.9E+11 | 7% | N/A |
| | G4 | 5336 | N/A | N/A | 8.5E+12 | | | | |
| | G5 | 2555 | N/A | N/A | 8.2E+12 | | | | |
| | G6 | 1280 | N/A | N/A | 8.2E+12 | | | | |
| | G7 | 657 | N/A | N/A | 8.4E+12 | | | | |
| | G8 | 324 | N/A | N/A | 8.3E+12 | | | | |
| | G9 | 174 | N/A | N/A | 8.9E+12 | | | | |
| | G10 | 97 | N/A | N/A | 1.0E+13 | | | | |
| | G11 | 45 | N/A | N/A | 9.3E+12 | | | | |

In Table 3, “Function” identifies whether the sample contained an unknown amount of CMVpr or a known amount as a positive control; “Conc. [cp/μl](dPCR reaction)” means the raw data from the digital PCR; “Expected Vector Genome Titer [vg/mL]” means the vector genome titer of the known samples; “Accuracy (% REC)” means the accuracy of the measurement relative to the Expected Vector Genome Titer [vg/mL]; “% REC” means percent recovery; “Vector Genome Titer [vg/mL]” means the vector genome titer of the undiluted sample which is calculated by multiplying the Conc. [cp/μl] by the dilution factor; “Average Vector Genome Titer [vg/mL]” means the average vector genome titer of the Vector Genome Titer [vg/mL] values used in the average; “Standard Deviation [vg/mL]” means the standard deviation of the values used to generate the average by using STDEV.S function in Excel; “Precision (% CV)” means the coefficient of variation calculated from the standard deviation and the average; and “AVG. Accuracy (% REC)” means the accuracy of the averaged measurement relative to the Expected Vector Genome Titer [vg/mL]. These results show that absolute quantification of the CMVpr genetic element using digital PCR was both accurate (known was within +/−30% recovery) and precise (less than 30% coefficient of variation in both the known and unknown samples) when tested in multiplex with eGFP and the idPCR AAV8 DNA Oligonucleotide of the Antibody-Oligonucleotide Conjugate.

TABLE 4
AAV vector genome titer absolute quantification using eGFP.

| | | AAV Vector Genome Titer for eGFP | | | | | | | |
|---|---|---|---|---|---|---|---|---|---|
| | | | Expected | | Vector | Average | | | |
| | | Conc. | Vector | | Genome | Vector | | | |
| Sample | | [cp/μL] | Genome | | Titer | Genome | Standard | | AVG. |
| Information | | (dPCR | Titer | Accuracy | [vg/mL] | Titer | Deviation | Precision | Accuracy |
| Function | Well | reaction) | [vg/mL] | (% REC) | (eGFP) | [vg/mL] | [vg/mL] | (% CV) | (% REC) |
| Known | C4 | 5535 | 7500 | 74% | 8.9E+12 | 8.7E+12 | 4.0E+11 | 5% | 73% |
| | C5 | 2707 | 3750 | 72% | 8.7E+12 | | | | |
| | C6 | 1301 | 1875 | 69% | 8.3E+12 | | | | |
| | C7 | 651 | 938 | 69% | 8.3E+12 | | | | |
| | C8 | 347 | 469 | 74% | 8.9E+12 | | | | |
| | C9 | 176 | 234 | 75% | 9.0E+12 | | | | |
| | C10 | 81 | 117 | 69% | 8.2E+12 | | | | |
| | C11 | 46 | 59 | 78% | 9.4E+12 | | | | |
| Unknown | G4 | 5139 | N/A | N/A | 8.2E+12 | 9.0E+12 | 8.1E+11 | 9% | N/A |
| | G5 | 2654 | N/A | N/A | 8.5E+12 | | | | |
| | G6 | 1325 | N/A | N/A | 8.5E+12 | | | | |
| | G7 | 687 | N/A | N/A | 8.8E+12 | | | | |
| | G8 | 325 | N/A | N/A | 8.3E+12 | | | | |
| | G9 | 176 | N/A | N/A | 9.0E+12 | | | | |
| | G10 | 101 | N/A | N/A | 1.0E+13 | | | | |
| | G11 | 50 | N/A | N/A | 1.0E+13 | | | | |

Table 4 shows the absolute quantification of AAV Vector Genome Titer determined by detecting and quantifying eGFP. These results show that absolute quantification of the eGFP reporter gene using digital PCR was precise (less than 30% coefficient of variation in both the known and unknown samples) but with lower accuracy than relative quantification (the known showed a recovery of 69%, outside of the +/−30% acceptance criteria) when tested in multiplex with CMVpr and the idPCR AAV8 DNA Oligonucleotide of the Antibody-Oligonucleotide Conjugate.

TABLE 5
AAV vector genome titer relative quantification using CMVpr.

| | | AAV Vector Genome Titer for CMVpr | | | | | | | | |
|---|---|---|---|---|---|---|---|---|---|---|
| | | | | Expected | | Calculated | Average | | | |
| | | Conc. | | Vector | | Vector | Vector | | | |
| Sample | | [cp/μL] | Calculated | Genome | | Genome | Genome | Standard | | AVG. |
| Information | | (dPCR | Conc. | Titer | Accuracy | Titer | Titer | Deviation | Precision | Accuracy |
| Function | Well | reaction) | [cp/μL] | [vg/mL] | (% REC) | [vg/mL] | [vg/mL] | [vg/mL] | (% CV) | (% REC) |
| Standard | C3 | 10468 | 14974 | 15000 | 100% | 1.2E+13 | 1.1E+13 | 8.8E+11 | 8% | 96% |
| | C4 | 5254 | 7511 | 7500 | 100% | 1.2E+13 | | | | |
| | C5 | 2615 | 3733 | 3750 | 100% | 1.2E+13 | | | | |
| | C6 | 1262 | 1797 | 1875 | 96% | 1.2E+13 | | | | |
| | C7 | 629 | 891 | 938 | 95% | 1.1E+13 | | | | |
| | C8 | 348 | 489 | 469 | 104% | 1.3E+13 | | | | |
| | C9 | 169 | 233 | 234 | 100% | 1.2E+13 | | | | |
| | C10 | 75 | 98 | 117 | 84% | 1.0E+13 | | | | |
| | C11 | 41 | 49 | 59 | 84% | 1.0E+13 | | | | |
| Unknown | G3 | 10790 | 15434 | N/A | N/A | 1.2E+13 | 1.2E+13 | 5.8E+11 | 5% | N/A |
| | G4 | 5336 | 7628 | N/A | N/A | 1.2E+13 | | | | |
| | G5 | 2555 | 3647 | N/A | N/A | 1.2E+13 | | | | |
| | G6 | 1280 | 1822 | N/A | N/A | 1.2E+13 | | | | |
| | G7 | 657 | 932 | N/A | N/A | 1.2E+13 | | | | |
| | G8 | 324 | 455 | N/A | N/A | 1.2E+13 | | | | |
| | G9 | 174 | 240 | N/A | N/A | 1.2E+13 | | | | |
| | G10 | 97 | 130 | N/A | N/A | 1.3E+13 | | | | |
| | G11 | 45 | 56 | N/A | N/A | 1.1E+13 | | | | |

Table 5 shows the relative quantification of AAV Vector Genome Titer determined by detecting and quantifying CMVpr. A standard curve, shown in FIG. 6 (Standard Curve for dPCR for AAV Vector Genome Titer for Target CMVpr), was prepared from quantifying the CMVpr of known standards. The CMVpr of unknown samples was determined by comparison to the standard curve. “Calculated Conc. [cp/μL]” means the concentration of the dPCR reaction, calculated from the standard curve and “Calculated Vector Genome Titer [vg/mL]” means the vector genome titer of the undiluted sample which is calculated by multiplying the Calculated Conc. [cp/μl] by the dilution factor. These results show that relative quantification of the CMVpr genetic element using digital PCR with a reference standard was both accurate (known was within +/−30% recovery) and precise (less than 30% coefficient of variation in both the known and unknown samples) when tested in multiplex with eGFP and the idPCR AAV8 DNA Oligonucleotide of the Antibody-Oligonucleotide Conjugate. Calibration of the digital PCR for CMVpr vector genome titer quantification using the same reference standard used for the idPCR AAV8 capsid titer assay leads to higher accuracy for both the CMVpr vector genome titer assay as well as the filling grade (% full capsids) calculated from these values.

TABLE 6
AAV vector genome titer relative quantification using eGFP.

| | | AAV Vector Genome Titer for eGFP | | | | | | | | |
|---|---|---|---|---|---|---|---|---|---|---|
| | | | | Expected | | Calculated | Average | | | |
| | | Conc. | | Vector | | Vector | Vector | | | |
| Sample | | [cp/μL] | Calculated | Genome | | Genome | Genome | Standard | | AVG. |
| Information | | (dPCR | Conc. | Titer | Accuracy | Titer | Titer | Deviation | Precision | Accuracy |
| Function | Well | reaction) | [cp/μL] | [vg/mL] | (% REC) | [vg/mL] | [vg/mL] | [vg/mL] | (% CV) | (% REC) |
| Standard | C4 | 5535 | 7544 | 7500 | 101% | 1.2E+13 | 1.2E+13 | 8.0E+11 | 7% | 102% |
| | C5 | 2707 | 3703 | 3750 | 99% | 1.2E+13 | | | | |
| | C6 | 1301 | 1795 | 1875 | 96% | 1.1E+13 | | | | |
| | C7 | 651 | 911 | 938 | 97% | 1.2E+13 | | | | |
| | C8 | 347 | 498 | 469 | 106% | 1.3E+13 | | | | |
| | C9 | 176 | 266 | 234 | 113% | 1.4E+13 | | | | |
| Unknown | G4 | 5139 | 7006 | N/A | N/A | 1.1E+13 | 1.2E+13 | 8.5E+11 | 7% | N/A |
| | G5 | 2654 | 3631 | N/A | N/A | 1.2E+13 | | | | |
| | G6 | 1325 | 1826 | N/A | N/A | 1.2E+13 | | | | |
| | G7 | 687 | 959 | N/A | N/A | 1.2E+13 | | | | |
| | G8 | 325 | 469 | N/A | N/A | 1.2E+13 | | | | |
| | G9 | 176 | 266 | N/A | N/A | 1.4E+13 | | | | |

Table 6 shows the relative quantification of AAV Vector Genome Titer determined by detecting and quantifying eGFP. A standard curve, shown in FIG. 7 (Standard Curve for dPCR for AAV Vector Genome Titer for Target eGFP), was prepared from quantifying the eGFP of known standards. The eGFP of unknown samples was determined by comparison to the standard curve. These results show that relative quantification of the eGFP reporter gene using digital PCR with a reference standard was both accurate (known was within +/−30% recovery) and precise (less than 30% coefficient of variation in both the known and unknown samples) when tested in multiplex with CMVpr and the idPCR AAV8 DNA Oligonucleotide of the Antibody-Oligonucleotide Conjugate. Calibration of the digital PCR for eGFP vector genome titer quantification using the same reference standard used for the idPCR AAV8 capsid titer assay leads to higher accuracy for both the CMVpr vector genome titer assay as well as the filling grade (% full capsids) calculated from these values.

TABLE 7
AAV8 capsid titer relative quantification using the DNA oligonucleotide
sequence of the antibody-oligonucleotide conjugate.

| | | AAV8 Capsid Titer | | | | | | | | |
|---|---|---|---|---|---|---|---|---|---|---|
| | | | | | | | Average | | | |
| | | Conc. | Expected | Calculated | | Calculated | Calculated | | | |
| Sample | | [cp/μL] | Capsid | Capsid | | Capsid | Capsid | Standard | | AVG. |
| Information | | (dPCR | Titer | Titer | Accuracy | Titer | Titer | Deviation | Precision | Accuracy |
| Function | Well | reaction) | [vp/μL] | [vp/μL] | (% REC) | [vp/mL] | [vp/mL] | [vp/mL] | (% CV) | (% REC) |
| Standard | C3 | 22186 | 4.6E+04 | 4E+04 | 80% | 3.0E+13 | 3.6E+13 | 3.5E+12 | 10% | 97% |
| | C4 | 13689 | 2.3E+04 | 2E+04 | 99% | 3.7E+13 | | | | |
| | C5 | 7083 | 1.2E+04 | 1E+04 | 102% | 3.8E+13 | | | | |
| | C6 | 3741 | 5.8E+03 | 6E+03 | 107% | 3.9E+13 | | | | |
| | C7 | 1850 | 2.9E+03 | 3E+03 | 103% | 3.8E+13 | | | | |
| | C8 | 958 | 1.4E+03 | 1E+03 | 102% | 3.8E+13 | | | | |
| | C9 | 458 | 7.2E+02 | 6E+02 | 87% | 3.2E+13 | | | | |
| Unknown | G3 | 22434 | 4.6E+04 | N/A | N/A | 3.0E+13 | 3.6E+13 | 4.0E+12 | 11% | N/A |
| | G4 | 14375 | 2.3E+04 | N/A | N/A | 3.9E+13 | | | | |
| | G5 | 7013 | 1.2E+04 | N/A | N/A | 3.7E+13 | | | | |
| | G6 | 3604 | 5.8E+03 | N/A | N/A | 3.8E+13 | | | | |
| | G7 | 1850 | 2.9E+03 | N/A | N/A | 3.8E+13 | | | | |
| | G8 | 953 | 1.4E+03 | N/A | N/A | 3.7E+13 | | | | |
| | G9 | 424 | 7.2E+02 | N/A | N/A | 2.9E+13 | | | | |

Table 7 shows the relative quantification of AAV8 Capsid Titer determined by detecting and quantifying the DNA oligonucleotide sequence of the antibody-oligonucleotide conjugate. For the DNA oligonucleotide target of the anti-AAV antibody-DNA oligonucleotide conjugate, a standard was used to create a standard curve shown in FIG. 8 (Standard Curve for dPCR for AAV8 Capsid Titer for Target AAV8 Antibody DNA Oligonucleotide of the Antibody-Oligonucleotide Conjugate) by plotting known capsid titer values to Ct values. The capsid titer of samples of interest was determined by comparison to this standard curve. The results shown in Table 7 show good accuracy (known was within +/−30% recovery) and precision (less than 30% coefficient of variation in both the known and unknown samples) of the idPCR AAV8 capsid titer assay when using an AAV8 reference standard with known capsid titer. These results confirm that the AAV capsid titer can be determined in a multiplex reaction with assays targeting the vector genome.

Alternatively, qPCR or ddPCR could be performed using known methods. For the AAV vector genome sequence target, the genome copy titer could be determined by absolute quantitation. For the DNA oligonucleotide target of the anti-AAV antibody-DNA oligonucleotide conjugate, a standard could be used to create a standard curve by plotting known capsid titer values to the absolute quantitation concentrations for this target. The capsid titer of samples of interest could be determined by comparison to this standard curve.

### Example 7

### Determination of Filling Grade (% Full Capsids)

The filling grade, which is the ratio of full to total AAV capsids (% full capsids), was determined as shown in Table 8. The average genome copy titer is divided by the average capsid titer. “Expected Filling Grade” means the filling grade of the known sample or standard and is calculated from digital PCR for AAV vector genome titer and an AAV8 capsid ELISA kit (Progen PRAAV8XP) results. “Accuracy (% REC)” means accuracy of the measurement relative to the Expected Filling Grade (% full capsids) and is determined by dividing the Filling Grade (% full capsids) by the Expected Filling Grade (% full capsids). These results show that this material and method allow for the simultaneous quantification of AAV vector genome titer and AAV8 capsid titer. By using the known sample as a reference standard for both AAV vector genome titer and AAV8 capsid titer, the resulting titers can be used to calculate a Filling Grade (% full capsids) result which shows accuracy equivalent to the conventional method (ELISA).

TABLE 8
Filling grade for AAV capsids.

| | | | | Expected | |
|---|---|---|---|---|---|
| | | | Filling | Filling | |
| | | | Grade | Grade | |
| | Average | | (% full | (% full | Accuracy |
| Result | Titer | Units | capsids) | capsids) | (% REC) |
| Absolute Quantification | 8.30E+12 | vg/mL | 23% | 32% | 71% |
| CMVpr Genome Titer | | | | | |
| Absolute Quantification | 8.70E+12 | vg/mL | 24% | 38% | 64% |
| eGFP Genome Titer | | | | | |
| Relative Quantification | 1.10E+13 | vg/mL | 31% | 32% | 94% |
| CMVpr Genome Titer | | | | | |
| Relative Quantification | 1.20E+13 | vg/mL | 33% | 38% | 88% |
| eGFP Genome Titer | | | | | |
| Relative Quantification | 3.60E+13 | vp/mL | N/A | N/A | N/A |
| AAV8 Capsid Titer | | | | | |

As will be understood by those familiar with the art, the present invention may be embodied in other specific forms without departing from the spirit or other essential characteristics thereof. Accordingly, the disclosures and descriptions herein are intended to be illustrative, but not limiting, of the scope of the invention that is set forth in the following claims.

I claim:

1. An antibody-DNA oligonucleotide conjugate comprising an antibody to a capsid of an AAV coupled to a DNA oligonucleotide; wherein the oligonucleotide is of sufficient length to be a target for a primer and probe for PCR.

2. The antibody-DNA oligonucleotide conjugate of claim 1 wherein the oligonucleotide includes a site for cleavage by a restriction endonuclease.

3. The antibody-DNA oligonucleotide conjugate of claim 2 wherein the restriction endonuclease is MspI.

4. The antibody-DNA oligonucleotide conjugate of claim 1 wherein the oligonucleotide is SEQ ID NO: 1.

5. The antibody-DNA oligonucleotide conjugate of claim 1 wherein the oligonucleotide has at least 90% sequence identity with SEQ ID NO: 1.

6. The antibody-DNA oligonucleotide conjugate of claim 1 wherein the oligonucleotide has least 70% sequence identity with SEQ ID NO: 1.

7. The antibody-DNA oligonucleotide conjugate of claim 1 wherein the oligonucleotide is single-stranded.

8. A method to determine an AAV capsid titer and an AAV genome copy titer in a single reaction comprising:
mixing a cell lysate containing an AAV of interest with paramagnetic beads bound with anti-AAV antibodies configured to bind the AAV of interest to the anti-AAV antibodies, such that the AAV of interest binds to the anti-AAV antibodies to form AAV-bound paramagnetic beads;
separating the AAV-bound paramagnetic beads from cell lysate solution;
immunostaining the AAV-bound paramagnetic beads with an antibody-DNA conjugate comprising an antibody to a capsid of an AAV coupled to a DNA oligonucleotide wherein the oligonucleotide is of sufficient length to be a target for a primer and probe set for PCR;
denaturing the AAV of interest with heat treatment;
performing multiplex PCR with the primer and probe set for amplification of the DNA oligonucleotide of the antibody-DNA oligonucleotide conjugate and a second primer and probe set for amplification of the AAV genomic sequence; and
determining the capsid titer and the genome copy titer of the AAV of interest.

9. The method of claim 8 wherein the oligonucleotide is SEQ ID NO: 1.

10. The method of claim 8 wherein the oligonucleotide has at least 90% sequence identity with SEQ ID NO: 1.

11. The method of claim 8 wherein the oligonucleotide has at least 70% sequence identity with SEQ ID NO: 1.

12. The method of claim 8 further comprising a plurality of primer and probe sets for amplification of a plurality of AAV genomic sequences.

13. The method of claim 8 wherein the multiplex PCR is qPCR.

14. The method of claim 8 wherein the multiplex PCR is dPCR.

15. The method of claim 10 wherein the genome copy titer and capsid titer are determined by absolute quantification.

16. The method of claim 10 wherein the genome copy titer and capsid titer are determined by relative quantification.

17. The method of claim 8 wherein the multiplex PCR is ddPCR.

18. The method of claim 8 wherein the DNA oligonucleotide is cleaved with a restriction endonuclease before performing multiplex PCR.

19. The method of claim 8 wherein the AAV of interest is AAV serotype 8.

20. The method of claim 8 further comprising determining the filling grade of the AAV capsid of interest.


SEQUENCE LISTING (10 sequences)

SEQ ID NO: 1 - DNA, 89 bp, synthetic construct
  source 1..89: mol_type = other DNA
  modified_base 1: mod_base = OTHER; note = Amino C6 modifier
accggcatgtgcaccgtgaagtcctccgctcccccctacaaagacgataagctagcagcattgcaaggatcggcgatattaacagagat

SEQ ID NO: 2 - DNA, 18 bp, synthetic construct
  source 1..18: mol_type = other DNA
gtgcaccgtgaagtcctc

SEQ ID NO: 3 - DNA, 21 bp, synthetic construct
  source 1..21: mol_type = other DNA
ctctgttaatatcgccgatcc

SEQ ID NO: 4 - DNA, 28 bp, synthetic construct
  source 1..28: mol_type = other DNA
cccctacaaagacgataagctagcagca

SEQ ID NO: 5 - DNA, 24 bp, synthetic construct
  source 1..24: mol_type = other DNA
ttcctacttggcagtacatctacg

SEQ ID NO: 6 - DNA, 21 bp, synthetic construct
  source 1..21: mol_type = other DNA
gtcaatggggtggagacttgg

SEQ ID NO: 7 - DNA, 25 bp, synthetic construct
  source 1..25: mol_type = other DNA
tgggcgtggatagcggtttgactca

SEQ ID NO: 8 - DNA, 24 bp, synthetic construct
  source 1..24: mol_type = other DNA
caacagccacaacgtctatatcat

SEQ ID NO: 9 - DNA, 21 bp, synthetic construct
  source 1..21: mol_type = other DNA
atgttgtggcggatcttgaag

SEQ ID NO: 10 - DNA, 25 bp, synthetic construct
  source 1..25: mol_type = other DNA
ccgacaagcagaagaacggcatcaa